(19) United States
(12) Patent Application Publication
(10) Pub. No.: US 2024/0272134 A1
(43) Pub. Date: Aug. 15, 2024

(54) SUSPENDED SELF-BALANCING SELF-CRUISING ONLINE WATER QUALITY MONITORING DEVICE, ONLINE WATER QUALITY MONITORING METHOD, AND ONLINE WATER QUALITY ASSESSMENT METHOD

(71) Applicant: JIANGSU UNIVERSITY, Zhenjiang (CN)

(72) Inventors: Yue JIANG, Zhenjiang (CN); You TANG, Zhenjiang (CN); Hong LI, Zhenjiang (CN); Lin HUA, Zhenjiang (CN); Jialing LIU, Zhenjiang (CN)

(73) Assignee: JIANGSU UNIVERSITY, Zhenjiang (CN)

(21) Appl. No.: 17/919,545

(22) PCT Filed: Feb. 10, 2022
(86) PCT No.: PCT/CN2022/075857

§ 371 Date: Oct. 18, 2022

(30) Foreign Application Priority Data
Jan. 10, 2022 (CN) 202210022629.0

(51) Int. Cl. G01N 33/18; B63B 35/00; G05D 1/646; G05D 107/00; G05D 109/30

(52) U.S. Cl. CPC G01N 33/18; B63B 35/00; G05D 1/646; B63B 2035/007; G05D 2107/25; G05D 2109/30

(57) ABSTRACT

A suspended self-balancing self-cruising online water quality monitoring device, an online water quality monitoring method, and an online water quality assessment method are provided. The device includes a suspension cabin main body, a communication and control system, and a suspended carrying platform configured for self-balancing attitude adjustment. Suspension feet configured to drive the suspension cabin main body to implement self-balancing attitude adjustment, cruising, or fixed-point suspension are arranged outside the suspension cabin main body. The communication and control system is configured to plan a W-shaped water-region cruising path and sampling points thereon according to a topography of a water region, control the water sample testing device to test quality of water along the sampling points online, assess water quality of the water region according to an online testing result, and output the water quality of the water region. The device achieves precise positioning and self-cruise monitoring are achieved.

### CROSS REFERENCE TO THE RELATED APPLICATIONS

This application is the national phase entry of International Application No. PCT/CN2022/075857, filed on Feb. 10, 2022, which is based upon and claims priority to Chinese Patent Application No. 202210022629.0, filed on Jan. 10, 2022, the entire contents of which are incorporated herein by reference.

### TECHNICAL FIELD

The present invention belongs to the technical field of environmental water quality monitoring, and particularly relates to a suspended self-balancing self-cruising online water quality monitoring device, an online water quality monitoring method, and an online water quality assessment method.

### BACKGROUND

With the increasing requirements for water environment quality, it is necessary to scientifically monitor and manage the water environment of river basins, assess and protect water ecosystems. In the prior art, a suspended water quality monitoring device adopts a water quality monitoring instrument fixedly arranged in the main body of the buoy as the core, and integrates a power supply and data transmission devices to form a monitoring system that can be deployed at a fixed point. Existing automated online water quality monitoring devices and methods are often suitable for fixed-point monitoring. When multiple sites in an area need to be monitored, multiple fixed-point monitoring devices are deployed respectively at the sites, and the water quality is assessed based on the testing data obtained at these separate sites. A water-borne self-cruising unmanned device in the prior art adopts a propulsion body such as a power component having fixed impellers on both sides or an unmanned aerial vehicle component. Such devices in the prior art have the following shortcomings. (1) When the water body has flowing water, the effects of fixed-point monitoring and assessment will be affected. Single-point detection is not comprehensive for water body assessment, while multi-point monitoring is not only costly but also is prone to assessment errors due to the independence of monitoring data obtained at separate sites. For a water region that requires continuous monitoring at multiple sites, it is difficult to accurately assess and reflect the overall comprehensive status of water quality in the water region. (2) It is impossible to plan optimal sampling points and the sample quantity according to the topography of the water region, which affects the adequate characterization of the water body status in the water body and the applicability of monitoring and assessment methods. (3) The water surface of most water regions is not in a static state for a long time, and the real-time online analysis of the water body often requires the water surface to remain relatively stable. The existing suspended and/or self-cruising unmanned water quality monitoring devices lack on-water attitude control and position control structures, making it difficult to achieve precise positioning, self-cruising, and a stable position. The monitoring result has certain errors due to the disturbance on the water surface. (4) The communication of monitoring data cannot visually reflect the results of water quality monitoring and assessment in the water region and give a warning when the water quality does not meet the quality standard, which affects the rapid expression of water quality assessment, early warning monitoring, and management efficiency. Therefore, it is necessary to design a corresponding technical solution to solve these technical problems.

### SUMMARY

The present invention aims to solve, at least to some extent, one of the above technical problems.

In view of this, the present invention provides a suspended self-balancing self-cruising online water quality monitoring device, an online water quality monitoring method, and an online water quality assessment method.

The following technical solutions are employed in the present invention.

A suspended self-balancing self-cruising online water quality monitoring device is provided, including a suspension cabin main body, a communication and control system, and a suspended carrying platform located inside the suspension cabin main body and configured for self-balancing attitude adjustment, where a plurality of suspension feet are arranged outside the suspension cabin main body; each of the suspension feet is configured to drive the suspension cabin main body to perform self-balancing attitude adjustment, cruising, or fixed-point suspension; a water sample testing device is arranged on the suspended carrying platform; and the communication and control system is configured to receive a satellite signal, plan a W-shaped water-region cruising path and sampling points on the W-shaped water-region cruising path according to a topography of a water region, control operation of the suspension feet, control attitude adjustment of the suspended carrying platform, control the water sample testing device to test quality of water outside the suspension cabin main body along the sampling points online, assess water quality of the water region according to an online testing result of the water sample testing device, and output a monitoring and assessment result.

In the above-mentioned suspended self-balancing self-cruising online water quality monitoring device, preferably, the suspension cabin main body includes a suspended bottom compartment having a changeable center of buoyancy and located at a bottom of the suspended carrying platform, and a cover connected to the suspended bottom compartment and located outside the suspended carrying platform; and the cover is configured to display a corresponding visual warning signal according to the monitoring and assessment result from the monitoring and control system.

In the above-mentioned suspended self-balancing self-cruising online water quality monitoring device, preferably, the communication and control system includes an external controller; the external controller includes a Global Positioning System (GPS) module, a communication module, and an external core controller configured to coordinate operation of the external controller; the GPS module is configured to communicate with a satellite through the communication module to feed back location information; the external core controller integrates an automatic W-shaped water-region cruising path planning algorithm; and the automatic W-shaped water-region cruising path planning algorithm is configured to solve and optimize the W-shaped water-region cruising path and the sampling points for the water sample testing device according to the location information from the GPS module, and optimize the W-shaped water-region cruising path to be a shortest path under a condition that the sampling points represent an overall status of the water region.

The external controller includes a first gyroscope and a first driving module; the first gyroscope is configured to analyze an attitude of the suspension cabin main body; the first driving module is configured to drive the suspension feet to implement attitude adjustment and operate; and the external core controller is configured to control operation of the first driving module according to a result of the automatic W-shaped water-region cruising path planning algorithm and/or an attitude feedback from the first gyroscope.

Each of the suspension feet includes a suspension leg and an ellipsoid suspension body; one end of the suspension leg is connected to the suspension cabin main body, and the other end of the suspension leg is connected to the ellipsoid suspension body; the suspension leg includes at least one joint; the joint is provided with a first driving mechanism connected to the first driving module; the first driving mechanism is configured to drive the joint to move relatively; a second driving mechanism is arranged between the ellipsoid suspension body and the suspension leg; and the second driving mechanism is connected to the first driving module and is configured to drive the ellipsoid suspension body to rotate relative to the suspension leg.

In the above-mentioned suspended self-balancing self-cruising online water quality monitoring device, preferably, the suspended carrying platform is rotatable relative to the suspension cabin main body; a balancing device configured to drive the suspended carrying platform to self-balance is arranged on the suspended carrying platform; the communication and control system includes an internal controller; the internal controller includes a second gyroscope, a second driving module, and an internal core controller configured to coordinate operation of the internal controller; the second gyroscope is configured to analyze an attitude of the suspended carrying platform; the second driving module is configured to drive the balancing device and the water sample testing device to operate; the internal core controller is configured to control the second driving module to drive the balancing device according to an attitude feedback from the second gyroscope; a water-region water quality assessment model is integrated on the internal core controller; and the water-region water quality assessment model is configured to assess the water quality of the water region according to the online testing result of the water sample testing device and generate the monitoring and assessment result;
the suspended carrying platform is rotatably connected to the suspension cabin main body through a spherical hinge connecting rod; the spherical hinge connecting rod includes a load-bearing rod, a spherical hinge ball shell, a spherical hinge ball, and a carrying-platform connecting rod; one end of the load-bearing rod is connected to a top of the suspension cabin main body, and the other end of the load-bearing rod is connected to the spherical hinge ball shell; the spherical hinge ball is arranged inside the spherical hinge ball shell and is rotatably fitted to an interior of the spherical hinge ball shell; one end of the carrying-platform connecting rod is connected to the spherical hinge ball, and the other end of the carrying-platform connecting rod is connected to the suspended carrying platform or the internal controller; the balancing device includes two balancing motors oppositely arranged on the suspended carrying platform; a motor shaft of each of the balancing motors is connected to a balancing flywheel;
the suspension cabin main body includes a fluorescent cover; a light-emitting diode (LED) warning light strip configured to output a corresponding light source according to the monitoring and assessment result from the communication and control system is arranged on the internal controller; the fluorescent cover is configured to emit a corresponding visual warning fluorescent signal under an excitation of the LED warning light strip; and the internal controller communicates with an Internet of Things device and outputs the monitoring and assessment result.

In the above-mentioned suspended self-balancing self-cruising online water quality monitoring device, preferably, the water sample testing device includes a water inlet pipeline, a testing chamber, a water quality sensor array, a water level sensor group, and a water outlet pipeline; the water inlet pipeline is communicated with an outside of the suspension cabin main body and the testing chamber; the testing chamber is configured to store a filtered water sample inputted through the water inlet pipeline; the water quality sensor array is configured to test the water sample in the testing chamber and transmit the online testing result including at least one type of parameters or at least one parameter; the water level sensor group is configured to feed back a water level of the water sample in the testing chamber to the communication and control system; the water outlet pipeline is communicated with the outside of the suspension cabin main body and the testing chamber; and the communication and control system is configured to control on/off of a water intake through the water inlet pipeline and on/off of a water output through the water outlet pipeline, and control operation of the water quality sensor array.

In the above-mentioned suspended self-balancing self-cruising online water quality monitoring device, preferably, a power supply system is arranged on the suspension cabin main body; the power supply system includes a solar panel arranged on the suspension cabin main body; and the communication and control system is configured to control the power supply system to supply power to the communication and control system, the suspension feet, and the water sample testing device.

An online water quality monitoring method based on the suspended self-balancing self-cruising online water quality monitoring device according to any one of the above implementations is provided, including:

S1: receiving, by the communication and control system, a satellite signal and obtaining a satellite topography data of a water region;

S2: calculating a longest diameter of a topography of the water region according to the satellite topography data, and setting two ends of the longest diameter as a starting shore point and a destination shore point, respectively;

S3: selecting several shore points on two sides of the longest diameter, and numbering the shore points alternately on the two sides starting from the starting shore point to the destination shore point, so that the shore points with adjacent numbers are connected to form an integer number of W-shaped patterns;

S4: calculating a characteristic region threshold according to the satellite topography data, where the characteristic region threshold is an upper limit of an area characterizing a property of the water region, optimizing positions and a number of the shore points based on whether an area of a region defined by lines connecting every three adjacent shore points of the shore points is less than or equal to the characteristic region threshold, and generating a W-shaped water-region cruising path planned according to the topography of the water region;

S5: selecting the sampling points on the W-shaped water-region cruising path;

S6: controlling, by the communication and control system, the suspension cabin main body to cruise along the sampling points;

S7: at each of the sampling points, controlling the suspension cabin main body to be suspended fixedly, and implementing a self-balancing attitude adjustment of the suspension cabin main body and the suspended carrying platform; and

S8: controlling the water sample testing device to test the quality of the water outside the suspension cabin main body online, generating and outputting an online monitoring result of the water quality of the water region.

In the above-mentioned online water quality monitoring method, preferably, the step S1-step S6 are based on an automatic W-shaped water-region cruising path planning algorithm; in the step S4, an area of a triangle defined by lines connecting every three adjacent shore points of the shore points is numbered sequentially based on the automatic W-shaped water-region cruising path planning algorithm, where the area of the triangle is Sⱼ, j is an integer from 1 to 4n−1, n is an integer equal to or greater than 1; a calculation formula of the characteristic region threshold M is M=3S_{water-region}/H*(4n−1), H=hₘ/hₗ, where S_{water-region} represents a total area of the water region in the satellite topography data in the step S1, hₘ represents a water depth at a deepest position of the water region in the satellite topography data in the step S1, and hₗ is a water depth at a shallowest position of the water region in the satellite topography data in the step S1; and the positions and the number of the shore points are optimized with all Sⱼ≤M to generate the W-shaped water-region cruising path planned according to the topography of the water region.

An online water quality assessment method based on the above-mentioned online water quality monitoring method is provided, including:

S9: setting, by the communication and control system, a water quality assessment criterion and an assessment factor;

S10: constructing a matrix including testing data of each of the sampling points according to the online monitoring result of the online water quality monitoring method;

S11: calculating a vector according to the matrix and the assessment factor; and

S12: generating and outputting a water quality assessment result of the water region according to a result of comparison between a norm of the vector and the water quality assessment criterion.

In the above-mentioned online water quality assessment method, preferably, the step S9-step S12 are based on a water-region water quality assessment model; a calculation formula of the assessment factor N of the water-region water quality assessment model in the step S9 is N=e*(1/k), where e represents a unit vector, and k represents a vector dimension of the matrix in the step S10; in the step S10, testing data obtained by the water sample testing device at an i^{th} sampling point is used as an m-dimensional vector Xᵢ, and a matrix M is constructed using k-dimensional Xᵢ to calculate a vector Xₒ, where a calculation formula of Xₒ is Xₒ=M*N; and in the step S12, a calculation formula of the norm is ∥X₀∥₂=λ, and the water quality assessment result of the water region is generated by comparing λ with the water quality assessment criterion.

Compared with the prior art, the present invention has the following beneficial effects.

(1) The suspended self-balancing self-cruising online water quality monitoring device has an automatic W-shaped water-region cruising path planning function; supports self-cruising fixed-point suspension, and self-balancing of the suspension cabin main body; supports self-balancing of the suspended carrying platform; and has an online water quality testing function, a water quality monitoring and assessment function, a communication function, a visual warning function of the suspension cabin main body, and a solar power supply function, thereby solving the problem of continuously optimizing the monitoring of the water region, the error problem caused by the disturbance on the water surface to the monitoring and assessment results, and the problem of early warning and monitoring of the monitoring and assessment results.

(2) The monitoring method based on the suspended self-balancing self-cruising online water quality monitoring device automatically plans a W-shaped water sampling and cruising path, to optimize the W-shaped water-region cruising path to be a shortest path under a condition that the sampling points fully represent an overall status of the water region, and facilitate the continuous mobile monitoring of different water regions, thereby solving the problem of continuously optimizing the monitoring of different waters.

(3) With the assessment method based on the monitoring method, the online monitoring results of all the sampling points in the water region and the assessment factor are fitted to obtain comprehensive assessment vector data of the water region, so as to comprehensively assess and analyze the overall status of the water region, thereby overcoming the incomprehensiveness of water body assessment.

To sum up, according to the technical solutions of the present invention, with the suspended self-balancing self-cruising online water quality monitoring device, the online water quality monitoring method, and the online water quality assessment method, a path and sampling points are automatically planned, precise positioning and self-cruise monitoring are achieved, the device remains stable throughout the monitoring process, and self-balancing of the device is not affected by the disturbance on the water surface, thereby improving the monitoring and assessment precision, and greatly reducing the cost of water-region monitoring and assessment, making the device especially suitable for the monitoring and assessment of water quality in different water regions that have flowing water and require continuous monitoring.

### BRIEF DESCRIPTION OF THE DRAWINGS

FIG. 1 is a schematic view of an overall structure of Embodiment 1 of the present invention.

FIG. 2 is a front view of Embodiment 1 of the present invention.

FIG. 3 is a top view of Embodiment 1 of the present invention.

FIG. 4 is a schematic structural view of a suspension foot according to Embodiment 1 of the present invention.

FIG. 5 is a structural cross-sectional view of a spherical hinge connecting rod according to Embodiment 1 of the present invention.

FIG. 6 is a three-dimensional structural view of the spherical hinge connecting rod according to Embodiment 1 of the present invention.

FIG. 7 is a top view showing a straight traveling state according to Embodiment 1 of the present invention.

FIG. 8 is a top view showing a turning state according to Embodiment 1 of the present invention.

FIG. 9 is a structural view of an external controller according to Embodiment 1 of the present invention.

FIG. 10 is a structural view of an internal controller according to Embodiment 1 of the present invention.

FIG. 11 is a schematic view showing an automatic W-shaped water-region cruising path planning algorithm flow and a planning process according to Embodiment 2 of the present invention.

FIG. 12 is a flowchart of a water-region water quality assessment model according to Embodiment 3 of the present invention.

List of reference numerals: suspension cabin main body 1, suspended bottom compartment 11, fluorescent cover 12; suspended carrying platform 2, external controller 31, internal controller 32, communication antenna 33, LED warning light strip 34; suspension foot 4, suspension leg 41, first joint 411, second joint 412, connecting rod 413, movable bracket 401, movable shaft 402, first transmission motor 403, transmission gear 404, sealing sheet 405, second transmission motor 406, ellipsoid suspended wood 42, threaded rod 43; spherical hinge connecting rod 5, load-bearing rod 51, spherical hinge ball shell 52, spherical hinge ball 53, carrying-platform connecting rod 54; water inlet pipeline 601, testing chamber 602, water quality sensor array 603, water level sensor group 604, water outlet pipeline 605, water inlet bottom valve 606, hose groove 607, water inlet through hole 608, sampling micro-pump 609, filter 610, upper water level monitoring sensor 6041, lower water level monitoring sensor 6042, water outlet micro-pump 611, water outlet through hole 612, sealing ring 613, sealing port 614; balancing motor 71, balancing flywheel 72, and solar panel 8.

### DETAILED DESCRIPTION OF THE EMBODIMENTS

Embodiments of the present invention will be exemplarily described in detail hereinafter with reference to accompanying drawings in which the same or like reference characters refer to the same or like elements or elements having the same or like functions throughout. The embodiments described below with reference to accompanying drawings are exemplary, and intended to explain, instead of limiting the present invention.

In the description of the present invention, it should be understood that the orientation or positional relationships indicated by the terms “center”, “longitudinal”, “transverse”, “length”, “width”, “thickness”, “upper”, “lower”, “axial”, “radial”, “vertical”, “horizontal”, “inner”, “outer”, etc. are based on the orientation or positional relationships shown in the drawings, and are only for the convenience of describing the present invention and simplifying the description, rather than indicating or implying that the apparatus or element described must have a specific orientation or be constructed and operated in a specific orientation, and therefore are not to be construed as limiting the present invention. Moreover, the terms “first” and “second” are used herein for purposes of description, and are not intended to indicate or imply relative importance or implicitly point out the number of the indicated technical feature. Therefore, the features defined by “first”, and “second” may explicitly or implicitly include one or more features. In the description of the present invention, “plural” means two or more, unless it is defined otherwise specifically. In the present invention, unless otherwise clearly specified and defined, the terms “mount”, “connect”, “couple”, “fix” and variants thereof should be interpreted in a broad sense, for example, may be a fixed connection, a detachable connection, or an integral connection; may be a mechanical connection or an electrical connection; or may be a direct connection, an indirectly connection via an intermediate medium, or communication between the interiors of two components. For those of ordinary skill in the art, the specific meanings of the above terms in the present invention can be understood according to specific circumstances.

### Embodiment 1

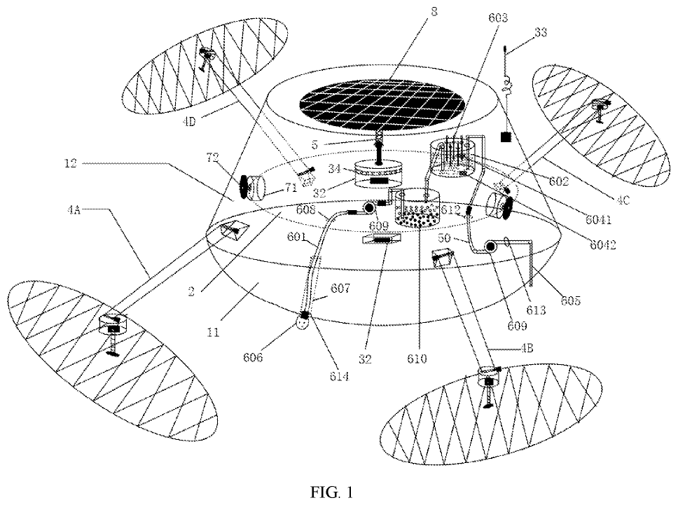

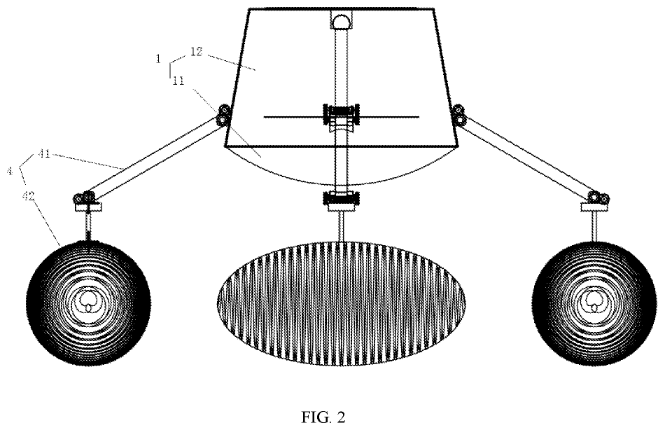

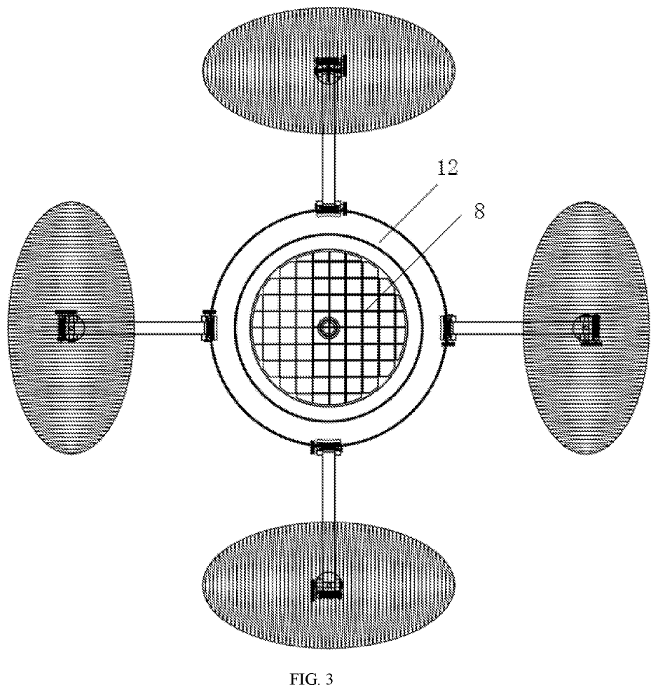

FIG. 1-FIG. 3 show a preferred implementation of the suspended self-balancing self-cruising online water quality monitoring device. The device includes a suspension cabin main body 1, a communication and control system, and a suspended carrying platform 2 located inside the suspension cabin main body 1 and configured for self-balancing attitude adjustment, where a plurality of suspension feet 4 are arranged outside the suspension cabin main body 1; each of the suspension feet 4 is configured to drive the suspension cabin main body 1 to implement self-balancing attitude adjustment, cruising, or fixed-point suspension; a water sample testing device is arranged on the suspended carrying platform 2; and the communication and control system is configured to receive a satellite signal, plan a W-shaped water-region cruising path and sampling points on the W-shaped water-region cruising path according to a topography of a water region, control operation of the suspension feet 4, control attitude adjustment of the suspended carrying platform 2, control the water sample testing device to test quality of water outside the suspension cabin main body 1 along the sampling points online, assess water quality of the water region according to an online testing result of the water sample testing device, and output a monitoring and assessment result.

In the above-mentioned suspended self-balancing self-cruising online water quality monitoring device, preferably, the suspension cabin main body 1 includes a suspended bottom compartment 11 having a changeable center of buoyancy and located at a bottom of the suspended carrying platform 2, and a cover connected to the suspended bottom compartment 11 and located outside the suspended carrying platform 2; and the cover is configured to display a corresponding visual warning signal according to the monitoring and assessment result from the monitoring and control system.

Preferably, the suspended bottom compartment 11 is of a hollow structure and is provided therein with ballast sand. The ballast sand may flow to cause the suspended bottom compartment 11 to change with the suspension cabin main body 1 to change its center of buoyancy, so as to realize the self-balancing attitude adjustment of the suspension cabin main body, thereby improving stability.

Preferably, the suspended bottom compartment 11 of the suspension cabin main body 1 is hemispherical-shaped to increase the buoyancy of the suspension cabin main body 1.

Preferably, the outer cover is connected to a top of the suspended bottom compartment 11.

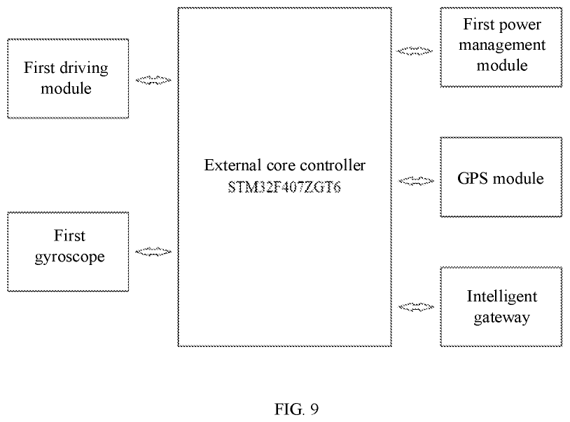

As shown in FIG. 1 and FIG. 9, In the above-mentioned suspended self-balancing self-cruising online water quality monitoring device, preferably, the communication and control system includes an external controller 31; the external controller 31 includes a Global Positioning System (GPS) module, a communication module, and an external core controller configured to coordinate operation of the external controller 31; the GPS module is configured to communicate with a satellite through the communication module to feed back location information; the external core controller integrates an automatic W-shaped water-region cruising path planning algorithm; and the automatic W-shaped water-region cruising path planning algorithm is configured to solve and optimize the W-shaped water-region cruising path and the sampling points for the water sample testing device according to the location information from the GPS module, and optimize the W-shaped water-region cruising path to be a shortest path under a condition that the sampling points represent an overall status of the water region.

Preferably, the communication module is an intelligent gateway, the suspension cabin main body 1 is equipped with a communication antenna 33, and the intelligent gateway sends and receives communication signals through the communication antenna 33 and communicates with a satellite, so that the GPS module communicates with the satellite through the communication module to feed back location information via a GPS satellite signal.

Preferably, the external controller 31 includes a first gyroscope and a first driving module; the first gyroscope is configured to analyze an attitude of the suspension cabin main body 1; the first driving module is configured to drive the suspension feet 4 to implement attitude adjustment and operate; and the external core controller is configured to control operation of the first driving module according to a result of the automatic W-shaped water-region cruising path planning algorithm and/or an attitude feedback from the first gyroscope.

Preferably, the external controller 31 is located at a center of an upper surface of the suspended bottom compartment 11, so that the center of buoyancy is stable.

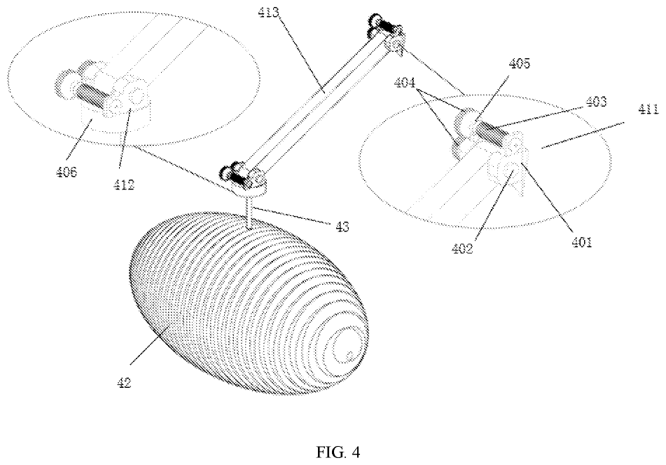

As shown in FIG. 1 to FIG. 4, preferably, each of the suspension feet 4 includes a suspension leg 41 and an ellipsoid suspension body; one end of the suspension leg 41 is connected to the suspension cabin main body 1, and the other end of the suspension leg 41 is connected to the ellipsoid suspension body; the suspension leg 41 includes at least one joint; the joint is provided with a first driving mechanism connected to the first driving module; the first driving mechanism is configured to drive the joint to move relatively; a second driving mechanism is arranged between the ellipsoid suspension body and the suspension leg 41; and the second driving mechanism is connected to the first driving module and is configured to drive the ellipsoid suspension body to rotate relative to the suspension leg 41.

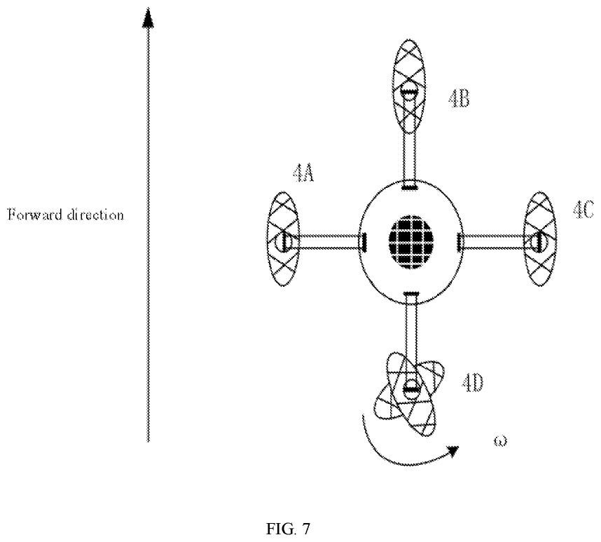

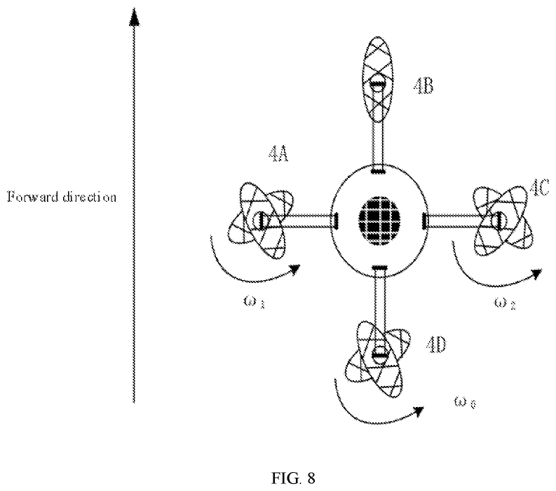

As shown in FIG. 1 to FIG. 4 and FIG. 7 to FIG. 8, preferably, there are four suspension feet 4, which are distributed on a front, rear and sides of the suspension cabin main body 1. the four suspension feet 4 are respectively suspension foot 4A, suspension foot 4B, suspension foot 4C, and suspension foot 4D, which are evenly distributed in four directions outside the suspension cabin main body 1 and are at 900 to each other.

Preferably, each of the suspension legs 41 includes a connecting rod 413 connected to the adjacent joint, the joint includes a movable bracket 401 and a movable shaft 402, the movable bracket 401 is connected to the suspension cabin main body 1 or two adjacent connecting rods 413 or second driving mechanisms, and the connecting rod 413 is rotatably connected to the movable bracket 401 through the movable shaft 402.

Preferably, the connecting rod 413 is of a hollow plastic structure, which is used for the electrical connection and wiring between the first driving mechanism and the second driving mechanism and the first driving module, and provides fixing and anti-corrosion functions. The connecting rod 413 is configured to rotate synchronously with the movable shaft 402.

Preferably, the first driving mechanism is a first transmission motor 403 mounted on the movable bracket 401 and electrically connected to the first driving module, the movable shaft 402 and a motor shaft of the first transmission motor 403 are each provided with a transmission gear 404, and the transmission gears 404 mesh with each other. The motor shaft of the first transmission motor 403 drives the adjacent movable shaft 402 and the connecting rod 413 under the transmission of the meshing transmission gear 404 to rotate on the movable bracket 401 about an axis of the movable shaft 402, so as to achieve relative movement of the joint through forward or reverse rotation of the first transmission motor 403.

Preferably, the joint includes two joints, a first joint 411 and a second joint 412. The first transmission motor 403 of the first joint 411 is arranged on or below the movable shaft 402. The movable bracket 401 of the first joint 411 is connected to the suspension cabin main body 1. The first transmission motor 403 of the second joint 412 is arranged on the left or right side of the movable shaft 402. The movable bracket 401 of the second joint 412 is connected to the second driving mechanism. In this way, two ends of the connecting rod 413 can both rotate 1800 in a plane perpendicular to the movable shaft 402 about the axis of the movable shaft 402 of the first joint 411 or the second joint 412, thereby realizing the angular adjustment between the suspension leg 41 and the suspension cabin main body 1 and the ellipsoid suspension body.

Preferably, a sealing sheet 405 is arranged between a motor body and the motor shaft of the first transmission motor 403. Sealing with the sealing sheet 405 can prevent water from entering the motor body to affect the operation.

Preferably, the first driving module is configured to control on/off of the first transmission motor 403.

Preferably, the second driving mechanism is a second transmission motor 406 mounted on the movable bracket 401 and electrically connected to the first driving module, and a motor shaft of the second transmission motor 406 is connected to the ellipsoid suspension body through a threaded rod 43. The ellipsoid suspension body is driven by the motor shaft of the second transmission motor 406 to rotate 360° in a horizontal plane.

Preferably, the first driving module is configured to control on/off and a rotational speed of the second transmission motor 406.

Preferably, the ellipsoid suspension body is an ellipsoid suspension wood 42 to ensure suspension and driving effects.

Preferably, a surface of the ellipsoid suspension body is provided with net-shaped threads. The net-shaped threads can increase the contact area between the ellipsoid suspension body and the water surface.

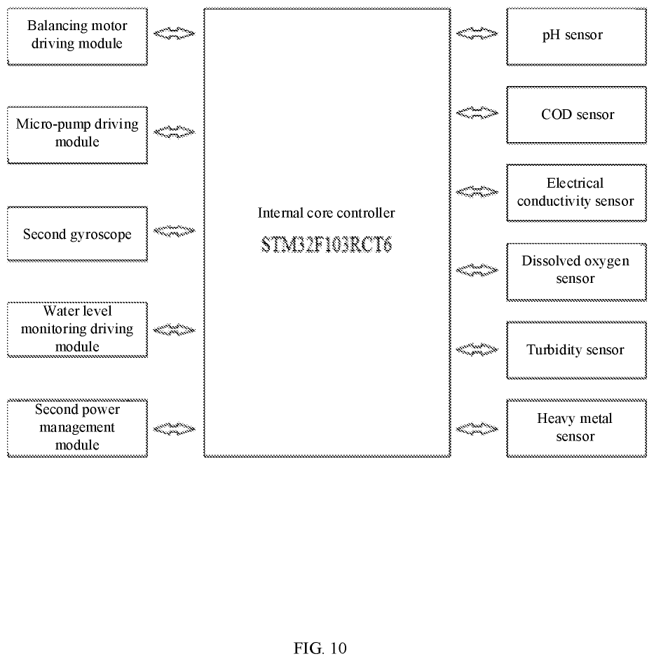

As shown in FIG. 1 and FIG. 10, in the above-mentioned suspended self-balancing self-cruising online water quality monitoring device, preferably, the suspended carrying platform 2 is rotatable relative to the suspension cabin main body 1; a balancing device configured to drive the suspended carrying platform 2 to self-balance is arranged on the suspended carrying platform 2; the communication and control system includes an internal controller 32; the internal controller 32 includes a second gyroscope, a second driving module, and an internal core controller configured to coordinate operation of the internal controller 32; the second gyroscope is configured to analyze an attitude of the suspended carrying platform 2; the second driving module is configured to drive the balancing device and the water sample testing device to operate; the internal core controller is configured to control the second driving module to drive the balancing device according to an attitude feedback from the second gyroscope; a water-region water quality assessment model is integrated on the internal core controller; and the water-region water quality assessment model is configured to assess the water quality of the water region according to the online testing result of the water sample testing device and generate the monitoring and assessment result.

Preferably, the suspended carrying platform 2 is of a disc-type structure.

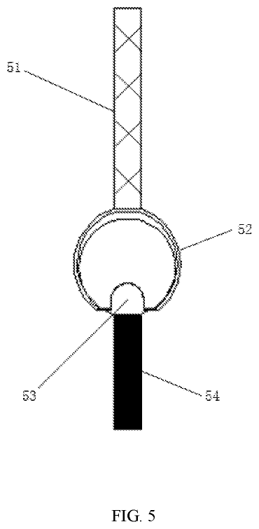

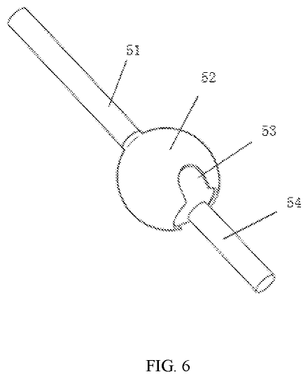

As shown in FIG. 1, FIG. 5 and FIG. 6, preferably, the suspended carrying platform 2 is rotatably connected to the suspension cabin main body 1 through a spherical hinge connecting rod 5.

Preferably, the spherical hinge connecting rod 5 includes a load-bearing rod 51, a spherical hinge ball shell 52, a spherical hinge ball 53, and a carrying-platform connecting rod 54; one end of the load-bearing rod 51 is connected to a top of the suspension cabin main body 1, and the other end of the load-bearing rod 51 is connected to the spherical hinge ball shell 52; the spherical hinge ball 53 is arranged inside the spherical hinge ball shell 52 and is rotatably fitted to an interior of the spherical hinge ball shell 52; one end of the carrying-platform connecting rod 54 is connected to the spherical hinge ball 53, and the other end of the carrying-platform connecting rod 54 is connected to the suspended carrying platform 2 or the internal controller 32.

Preferably, the carrying-platform connecting rod 54 can freely rotate about the spherical hinge ball shell within a spatial range of −30° to −90° with respect to the horizontal plane.

Preferably, the spherical hinge ball shell 52 is provided with a notch configured to limit the carrying-platform connecting rod 54. The carrying-platform connecting rod 54 can freely rotate about the spherical hinge ball shell within a spatial range of −30° to −90° with respect to the horizontal plane.

Preferably, the balancing device includes two balancing motors 71 oppositely arranged on the suspended carrying platform 2; a motor shaft of each of the balancing motors 71 is connected to a balancing flywheel 72. Through vector control of the balancing motor 71 by the second driving module, the balancing flywheel 72 can realize the self-balancing attitude adjustment of the suspended carrying platform 2, so that the body of the suspended carrying platform 2 is always in a suspended balanced state, and the suspended carrying platform 2 does not tilt along with shaking of the suspension cabin main body 1.

Preferably, the outer cover of the suspension cabin main body 1 is a fluorescent cover 12; a light-emitting diode (LED) warning light strip 34 configured to output a corresponding light source according to the monitoring and assessment result from the communication and control system is arranged on the internal controller 32 of the communication and control system; and the fluorescent cover 12 is configured to emit a corresponding visual warning fluorescent signal under an excitation of the LED warning light strip 34.

Preferably, the LED warning light strip 34 includes a light source configured to excite the fluorescent cover 12 to display three colors including red, orange, and green.

Preferably, the internal controller 32 is of a columnar structure, so that light from the light source of the LED warning light strip 34 is evenly distributed.

As shown in FIG. 1, in the above-mentioned suspended self-balancing self-cruising online water quality monitoring device, preferably, the water sample testing device includes a water inlet pipeline 601, a testing chamber 602, a water quality sensor array 603, a water level sensor group 604, and a water outlet pipeline 605; the water inlet pipeline 601 is communicated with an outside of the suspension cabin main body 1 and the testing chamber 602; the testing chamber 602 is configured to store a filtered water sample inputted through the water inlet pipeline 601; the water quality sensor array 603 is configured to test the water sample in the testing chamber 602 and transmit the online testing result including at least one type of parameters or at least one parameter; the water level sensor group 604 is configured to feed back a water level of the water sample in the testing chamber 602 to the communication and control system; the water outlet pipeline 605 is communicated with the outside of the suspension cabin main body 1 and the testing chamber 602; and the communication and control system is configured to control on/off of a water intake through the water inlet pipeline 601 and on/off of a water output through the water outlet pipeline 605, and control operation of the water quality sensor array 603.

Preferably, the water inlet pipeline 601 is of a hose structure, and the hose structure ensures the flexibility of water inlet. A water inlet bottom valve 606 is arranged at an end of the water inlet pipeline 601 extending to the outside of the suspended bottom compartment 11. A hose groove 607 configured for arranging the water inlet pipeline 601 is formed through the suspended bottom compartment 11. The suspended carrying platform 2 is provided with a water inlet through hole 608 configured for arranging the water inlet pipeline 601. A sampling micro-pump 609 connected to the second driving module is arranged on the water inlet pipeline 601. A water outlet of the sampling micro-pump 609 is connected to a water inlet of a filter 610 through the water inlet pipeline 601. A water outlet of the filter 610 is connected to the testing chamber 602 through the water inlet pipeline 601.

Preferably, the water inlet bottom valve 606 is threadedly connected to the hose groove 607 through a sealing port 614, and a seal is provided inside the water inlet bottom valve 606 to prevent water from entering the interior of the suspension cabin main body 1 to cause damage.

Preferably, the testing chamber 602 and the filter 610 are axially symmetrically distributed about a center line of the suspended carrying platform 2, so as to realize a weight balance of components disposed on the suspended carrying platform 2.

Preferably, a filter element of the filter 610 is a shell-ceramic ring structure. The filter element can filter impurities including sediment in the sampled water sample in the water inlet pipeline 601, so as to prevent a large amount of sediment from interfering with the detection or damaging the testing chamber 602, and filter out sediment in the water sample without introducing new ingredients.

Preferably, the water quality sensor array 603 is configured to detect and transmit various parameters including but not limited to pH, chemical oxygen demand (COD), electrical conductivity, dissolved oxygen, turbidity, and heavy metal content. The selection of the water quality sensor array 603 is configured according to requirements. The water quality sensor array 603 includes, but is not limited to, one or more of a pH sensor, a COD sensor, an electrical conductivity sensor, a dissolved oxygen sensor, a turbidity sensor, or a heavy metal sensor.

Preferably, the water level sensor group 604 includes an upper water level monitoring sensor 6041 and a lower water level monitoring sensor 6042 installed in an up part and a lower part of the testing chamber 602 respectively, and the upper water level monitoring sensor 6041 and the lower water level monitoring sensor 6042 are configured to detect the water level of the water sample in the testing chamber 602 and transmit the water level to the internal controller 32.

Preferably, the water outlet pipeline 605 is of a hose structure, and the hose structure ensures the flexibility of water outlet. A water outlet micro-pump 611 connected to the second driving module is arranged on the water outlet pipeline 605. The suspended carrying platform 2 is provided with a water outlet through hole 612 configured for arranging the water outlet pipeline 605. An end of the water outlet pipeline 605 extends to the outside of the suspension cabin main body 11. A sealing ring 613 is arranged between the water outlet pipeline and the suspension cabin main body 11 for sealing to prevent water from entering the interior of the suspension cabin main body 1.

As shown in FIG. 10, preferably, the second driving module includes a micro-pump driving module, a water level monitoring driving module, a balancing motor driving module, and a water quality sensor driving module. The micro-pump driving module is configured to drive, at sampling points solved by the automatic W-shaped water-region cruising path planning algorithm, the sampling micro-pump 609 and the water outlet micro-pump 611 to turn on or off according to water level information fed back by the upper water level monitoring sensor 6041 and the lower water level monitoring sensor 6042, so as to realize control of the sampling and the water level of the filtered water sample in the testing chamber 602. The water level monitoring driving module is configured to drive the upper water level monitoring sensor 6041 and the lower water level monitoring sensor 6042 to operate. The balancing motor driving module is configured to drive the balancing motor 71 to operate. The water quality sensor driving module is configured to drive the water quality sensor array 603 to operate.

As shown in FIG. 1, FIG. 9 and FIG. 10, in the above-mentioned suspended self-balancing self-cruising online water quality monitoring device, preferably, a power supply system is arranged on the suspension cabin main body 1; the power supply system includes a solar panel 8 arranged on the suspension cabin main body 1; and the communication and control system is configured to control the power supply system to supply power to the communication and control system, the suspension feet 4, and the water sample testing device.

Preferably, the solar panel 8 is arranged on the top of the suspension cabin main body 1, and the solar panel is configured to convert solar energy into electrical energy and store the electrical energy to supply power to the communication and control system, the suspension feet 4, the balancing device, the water sample testing device, and the LED warning light strip 34. The external controller 31 includes a first power management module configured to control the power supply system to distribute power to the external controller 31 and the suspension feet 4.

The internal controller 32 includes a second power management module configured to control the power supply system to distribute power to the internal controller 32, the balancing device, the water sample testing device, and the LED warning light strip 34.

Preferably, the external core controller is STM32F407ZGT6, and the external core controller coordinates operation of the first power management module, the GPS module, the communication module, the first gyroscope, and the first driving module.

Preferably, the internal core controller is STM32F103RCT6, and the internal core controller coordinates operation of the second power management module, the second gyroscope, and the second driving module.

Preferably, the internal controller 32 controls the operation of the second driving module according to the automatic W-shaped water-region cruising path planning algorithm of the external controller 31, and the water-region water quality assessment model communicates with an Internet of Things device through the intelligent gateway of the external controller 31 and the communication antenna 33, and outputs the monitoring and assessment result. The internal controller 32 and the external controller 31 are respectively arranged for internal and external environments of the device, and implement control independently of each other.

Preferably, the Internet of Things device includes a personal computer (PC)-side host computer, and the PC-side host computer is configured for a remote monitoring system to communicate with the suspended self-balancing self-cruising online water quality monitoring device to monitor and manage the monitoring and assessment result.

The above suspended self-balancing self-cruising online water quality monitoring device has the following functions.

(1) Automatic W-shaped water-region cruising path planning function: The external controller 31 communicates with a satellite through the GPS module to feed back the location information via a GPS satellite signal and obtain satellite topography data of the water region where the suspension cabin main body 1 is located. The automatic W-shaped water-region cruising path planning algorithm is used to solve and optimize the W-shaped water-region cruising path and the sampling points, which are sampling points of the water sample testing device. Therefore, an optimal cruising path is planned according to the topographical characteristics of the water region, so that the sampling points on this path can fully characterize the water quality characteristics of different positions in the water region.

(2) Self-cruising function of the suspension cabin main body 1: The external controller 31 controls the first driving module to drive the second driving mechanism of each suspension foot 4 according to the sampling points on the W-shaped water-region cruising path optimized by the automatic W-shaped water-region cruising path planning algorithm, to cause the ellipsoid suspension body to rotate relative to the suspension leg 41. When the ellipsoid suspension bodies of the suspension feet 4A, 4B, and 4C are oriented toward the same direction, and the ellipsoid suspension body of the suspension foot 4D rotates at a speed of ω, the device is pushed to advance. When the ellipsoid suspension body of the suspension foot 4B is kept in a straight-forward state, the ellipsoid suspension body of the suspension foot 4D rotates at a speed of coo to provide forward power, the ellipsoid suspension body of the suspension foot 4A rotates at a speed of ω₁ to provide a turning driving force, and the ellipsoid suspension body of the suspension foot 4C rotates at a speed of ω₂ to provide a turning driving force. When ω₁<ω₂, the device turns left; when ω₁>ω₂, the device turns right. Whereby, the self-cruising of the suspension cabin main body 1 along the sampling points is realized.

(3) Fixed-point suspension function of the suspension cabin main body 1: The external controller 31 drives the second driving mechanism of each suspension foot 4 through the first driving module according to the automatic W-shaped water-region cruising path planning algorithm and the location information fed back by the GPS module, to cause the ellipsoid suspension body to rotate relative to the suspension leg 41. When the ellipsoidal suspension bodies of the suspension foot 4A, the suspension foot 4B, the suspension foot 4C, and the suspension foot 4D rotate synchronously, fixed-point suspension of the suspension cabin main body 1 at the sampling point is realized.

(4) Self-balancing function of the suspension cabin main body 1: The external controller 31 analyzes and feeds back the attitude of the suspension cabin main body 1 through the first gyroscope, and the first driving module drives the relative movement of the joints of the suspension legs 41 through the second driving mechanism to adjust the attitude of the suspension feet 4, so that the self-balancing attitude adjustment of the suspension cabin main body 1 is realized under the support of the ellipsoid suspension bodies of the suspension feet 4.

(5) Self-balancing function of the suspended carrying platform 2: The suspended carrying platform 2 is suspended in and rotatably connected to the suspended cabin body 1 through the spherical hinge connecting rod 5. The internal controller 32 analyzes and feeds back the attitude of the suspended carrying platform 2 through the second gyroscope. The balancing motor driving module of the second driving module controls vectors of the balancing motor 71 of the balancing device to realize the self-balancing attitude adjustment of the suspended carrying platform 2 through the balancing flywheel 72, so that the internal and external environments of the device are separated from each other, and the suspended carrying platform 2 does not float along the floating of the suspension cabin main body 1.

(6) Online water quality testing function: When the suspension cabin main body 1 is fixedly suspended at a corresponding sampling point on the W-shaped water-region cruising path, and the suspension cabin main body 1 and the suspended carrying platform 2 achieve self-balancing, the internal controller 32 controls the sampling micro-pump 609 on the water inlet pipeline 601 to turn on through the micro-pump driving module of the second driving module, and the water inlet bottom valve 606 is automatically opened under the action of a pressure difference in the pipeline, so that the water sample outside the suspension cabin main body 1, i.e., on an outer side of the suspended bottom compartment 11, flows into the water inlet pipeline 601, is filtered by the filter 610, and enters the testing chamber 602 for storage.

The internal controller 32 uses the water level monitoring driving module of the second driving module to cause the upper water level monitoring sensor 6041 and the lower water level monitoring sensor 6042 in the water level sensor group 604 to feed back a water level of the testing chamber 602 respectively. When the water level of the water sample is higher than a position detected by the upper water level monitoring sensor 6041, the internal controller 32 uses the micro-pump driving module of the second driving module to control the water outlet micro-pump 611 on the water outlet pipeline 605 to turn on, so as to pump out the water sample in the testing chamber 602 through the water outlet pipeline 605. When the water level of the water sample is lower than a position detected by the lower water level monitoring sensor 6042, the internal controller 32 uses the micro-pump driving module of the second driving module to control the water outlet micro-pump 611 on the water outlet pipeline 605 to turn off, and controls the amount of water sampled each time in the testing chamber 602 by controlling on/off of the sampling micro-pump 609 and the water outlet micro-pump 611, thereby ensuring the reliability of testing by the water quality sensor array 603.

The internal controller 32 drives the water quality sensor array 603 that includes, but is not limited to, a pH sensor, a COD sensor, an electrical conductivity sensor, a dissolved oxygen sensor, a turbidity sensor, and a heavy metal sensor, through the water quality sensor driving module of the second driving module, to normatively test the water sample in the testing chamber 602 and transmit the online testing result including at least one type of parameters or at least one parameter, so as to realize the online testing of water quality at each sampling point.

(7) Water quality monitoring and assessment function: The internal core controller of the internal controller 32 assesses the water quality of the water region according to the online testing result of the water sample testing device by using the water-region water quality assessment model, and generates a monitoring and assessment result.

(8) Communication function: The external controller 31 communicates with a satellite through the GPS module so as to plan the W-shaped water-region cruising path and the sampling points on the W-shaped water-region cruising path according to the topography of the water region, and controls the suspension feet 4 to operate and drive the suspension cabin main body 1 to cruise or suspend at a fixed point. The monitoring and assessment result of the water-region water quality assessment model is communicated to the Internet of Things device including the PC-side host computer through the intelligent gateway of the external controller 31 and the communication antenna 33, so that the monitoring and assessment result of the water region can be remotely monitored and managed.

(9) Visual warning function of the suspension cabin main body 1: The internal controller 32 controls the LED warning light strip 34 to output the corresponding light source according to the monitoring and assessment result of the water quality assessment model, so that the fluorescent cover 12 emits a visual warning fluorescent signal including but not limited to red, orange, and green under the excitation of the LED warning light strip 34, thereby realizing a visual warning of the suspension cabin main body 1.

(10) Solar power supply function: When the power supply system including the solar panel 8 supplies power, the external controller 31 controls the distribution of power to the external controller 31 and the suspension feet 4 through the first power management module, and the internal controller 32 controls the power supply system to distribute power to the internal controller 32, the balancing device, the water sample testing device, and the LED warning light strip 34. The use of clean solar energy can save energy and reduce emission for the suspended self-balancing self-cruising online water quality monitoring device.

### Embodiment 2

A preferred implementation of the online water quality monitoring method is provided, which is based on the suspended self-balancing self-cruising online water quality monitoring device described in Embodiment 1. The method includes the following steps.

S1: The communication and control system receives a satellite signal and obtains a satellite topography data of a water region.

S2: A longest diameter of a topography of the water region is calculated according to the satellite topography data, and two ends of the longest diameter are set as a starting shore point and a destination shore point.

S3: Several shore points on two sides of the longest diameter are selected, and the shore points are numbered alternately on the two sides starting from the starting shore point to the destination shore point, so that the shore points with adjacent numbers are connected to form an integer number of W-shaped patterns.

S4: A characteristic region threshold is calculated according to the satellite topography data, where the characteristic region threshold is an upper limit of an area characterizing a property of the water region, positions and a number of the shore points are optimized based on whether an area of a region defined by lines connecting every three adjacent shore points is less than or equal to the characteristic region threshold, and a W-shaped water-region cruising path planned according to the topography of the water region is generated.

S5: The sampling points are selected on the W-shaped water-region cruising path.

S6: The communication and control system controls the suspension cabin main body 1 to cruise along the sampling points.

S7: At each of the sampling points, the suspension cabin main body 1 is controlled to be suspended fixedly, and a self-balancing attitude adjustment of the suspension cabin main body 1 and the suspended carrying platform 2 is implemented.

S8: The water sample testing device is controlled to test quality of water outside the suspension cabin main body 1 online, generate and output an online monitoring result of water quality of the water region.

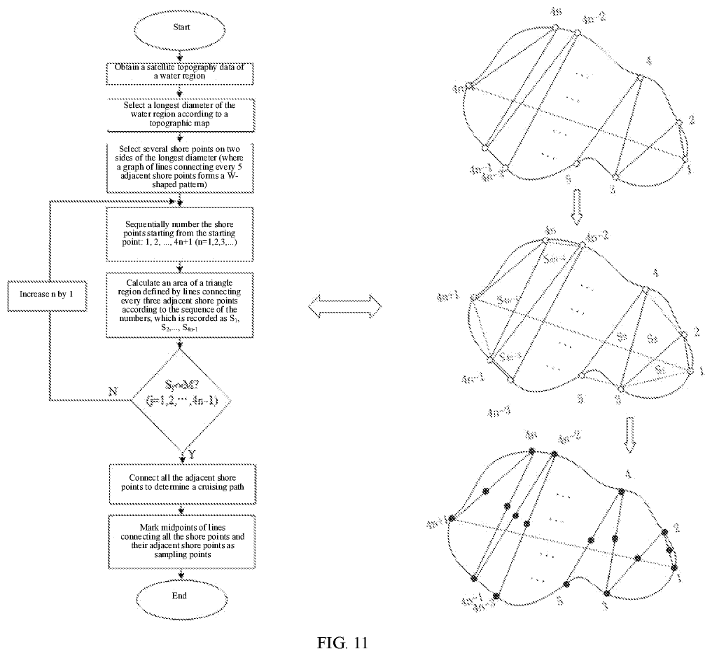

Preferably, the steps S1-S6 are based on the automatic W-shaped water-region cruising path planning algorithm of the external controller 31, and the automatic W-shaped water-region cruising path planning algorithm is shown in FIG. 11.

Preferably, in the step S3, based on the automatic W-shaped water-region cruising path planning algorithm, every 5 adjacent shore points form a W-shaped pattern, the starting shore point is numbered 1, the destination shore point is numbered 4n+1, and n is an integer equal to or greater than 1.

Preferably, in the step S4, an area of a triangle defined by lines connecting every three adjacent shore points is numbered sequentially based on the automatic W-shaped water-region cruising path planning algorithm, where the area of the triangle is Sⱼ, j is an integer from 1 to 4n−1, n is an integer equal to or greater than 1, and Sⱼ is recorded as S₁, S₂, . . . , S₄ₙ₋₁.

Preferably, in the step S4, the characteristic region threshold is calculated according to the satellite topography data by using the automatic W-shaped water-region cruising path planning algorithm. A calculation formula of the characteristic region threshold M is M=3S_{water-region}/H*(4n−1), H=hₘ/hₗ, where S_{water-region} represents a total area of the water region in the satellite topography data in the step S1, hₘ represents a water depth at a deepest position of the water region in the satellite topography data in the step S1, and hₗ is a water depth at a shallowest position of the water region in the satellite topography data in the step S1. The meaning of the calculation formula lies in that: 3S_{water-region} defines the sample characteristics of the water region, H is a characteristic factor calculated according to profile fault characteristics of the water region, 4n−1 is the total number of areas of triangles, an average area characteristic region threshold of a single triangle water-region area with respect to 3 times the total area of the water region is calculated, and H is used to correct the influence of different water depth distributions of the water region on the testing result of the water region, so that the characteristic region threshold is an upper limit of an area characterizing a property of the water region.

Preferably, in the step S4, it is determined whether Sⱼ is less than or equal to the characteristic region threshold M through sequential calculation using the automatic W-shaped water-region cruising path planning algorithm according to the triangle area number. When Sⱼ≤M, the sequential calculation is continuously performed, when Sⱼ>M, n is increased by 1 and the process goes back to the previous step of determining whether Sⱼ is less than or equal to the characteristic region threshold M through calculation, until all Sⱼ≤M. Thus, the optimized positions and number of the shore points are obtained, and the W-shaped water-region cruising path planned according to the topography of the water region is generated. The meaning of optimization calculation lies in that: setting Sⱼ to be less than or equal to the characteristic region threshold M can prevent some regions from being too large in the algorithm planning process and losing the function of characterizing the property of the water region.

Preferably, the calculation of the characteristic region threshold according to the satellite topography data by using the automatic W-shaped water-region cruising path planning algorithm in the step S4 and the sampling point in the step S5 may be based on the Technical Regulation of Water Quality Sampling of the People's Republic of China Industrial [SL187-96].

Preferably, in the step S5, the automatic W-shaped water-region cruising path planning algorithm is used to select midpoints of lines connecting all the shore points and their adjacent shore points with adjacent numbers on the W-shaped water-region cruising path as the sampling points, and the path planning ends.

Preferably, the online monitoring results in the step S8 are stored in the internal core controller.

### Embodiment 3

A preferred implementation of the online water quality assessment method is provided, which is based on the online water quality monitoring method described in Embodiment 2. The assessment method includes the following steps.

S9: The communication and control system sets a water quality assessment criterion and an assessment factor.

S10: A matrix including testing data of each of the sampling points is constructed according to the online monitoring result of the monitoring method.

S11: A vector is calculated according to the matrix and the assessment factor.

S12: A water quality assessment result of the water region is generated and outputted according to a result of comparison between a norm of the vector and the water quality assessment criterion.

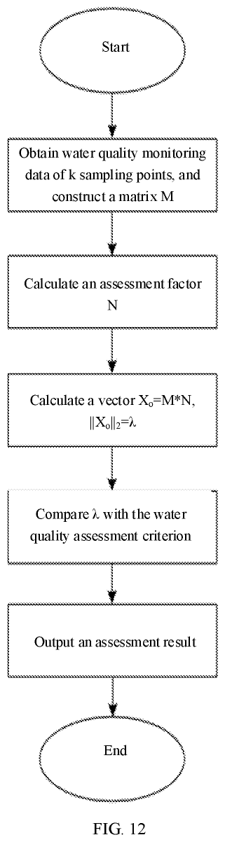

Preferably, the steps S9-S12 are based on the water-region water quality assessment model of the internal controller 32, and a flow of the water-region water quality assessment model is shown in FIG. 12.

Preferably, the water quality assessment criterion of the water-region water quality assessment model in the step S9 may be based on GB 3838-2002 “Environmental quality standards for surface water”.

Preferably, the water quality assessment criterion of the water quality assessment model in the step S9 may be based on the farmland water quality irrigation standard GB 5084-2021.

Preferably, the water quality assessment criterion of the water quality assessment model in the step S9 is to determine the water quality within a range of 0 to 2000.072 as excellent, to determine the water quality within a range of 2000.072 to 40000.01 as slightly polluted, and to determine the water quality within a range of 40000.01 or more as heavily polluted.

Preferably, a calculation formula of the assessment factor N of the water-region water quality assessment model in the step S9 is N=e*(1/k), where e represents a unit vector, and k represents a vector dimension of the matrix in the step S10.

Preferably, in the step S10, when the matrix is constructed with the water-region water quality assessment model, parameter types in the testing data of the water sample testing device are counted as y out of m, where m is an integer equal to or greater than 1; testing data obtained by the water sample testing device at an i^{th} sampling point is recorded as Xᵢ, where i=1, 2, 3, . . . , k, k is an integer equal to or greater than 1, and Xᵢ is an m-dimensional vector; a matrix M including the testing data of each sampling point is constructed, where the matrix M is a m*k-dimensional matrix.

Preferably, in the step S11, when the vector Xₒ is calculated using the water-region water quality assessment model, a calculation formula of Xₒ is Xₒ=M*N, where M is the matrix including the testing data of each sampling point, and N is the assessment factor.

Preferably, in the step S12, when the norm of the vector Xₒ is calculated using the water-region water quality assessment model, a 2-norm of Xₒ is calculated: ∥Xₒ∥₂=λ, and the water quality assessment result of the water region is generated by comparing λ with the water quality assessment criterion.

Preferably, in the step S12, when λ is within the range of 0 to 2000.072 and the water quality is determined to be excellent based on the water quality assessment model, the fluorescent cover 12 is shown as green, indicating that the water quality reaches a discharging criterion.

Preferably, in the step S12, when λ is within the range of 2000.072 to 40000.01 and the water quality is determined to be slightly polluted based on the water quality assessment model, the fluorescent cover 12 is shown as orange; indicating that the water quality index is about to reach the discharging standard.

Preferably, in the step S12, when λ is greater than or equal to 40000.01 and the water quality is determined to be heavily polluted based on the water quality assessment model, the fluorescent cover 12 is shown as red, indicating that the water quality does not reach the discharging standard.

Preferably, in the step S12, the water quality assessment result of the water region is outputted by an Internet of Things device through communication.

The beneficial effects of the above-mentioned suspended self-balancing self-cruising online water quality monitoring device, online water quality monitoring method, and online water quality assessment method are as follows.

(1) The suspended self-balancing self-cruising online water quality monitoring device solves the problem of continuously optimizing the monitoring of the water region, the error problem caused by the disturbance on the water surface to the monitoring and assessment results, and the problem of early warning and monitoring of the monitoring and assessment results.

One of the features of the device lies in that through the communication between the communication and control system and a satellite, the automatic W-shaped water-region cruising path planning algorithm can automatically plan and optimize the W-shaped water-region cruising path according to the topography of the water region and select sampling points on the W-shaped water-region cruising path; the suspension feet 4 drive the suspension cabin main body 1 to cruise or suspend at a fixed point for optimal sampling, so as to achieve precise positioning, self-cruising, and a stable testing position, thereby realizing continuous monitoring of water regions.

A second feature of the device lies in that in the case of disturbance on the water surface, the device can maintain fixed-point suspension at a specific sampling point, the center of buoyancy of each suspension cabin main body 1 is changeable, and the suspension cabin main body 1 is driven by the suspension feet 4 to quickly achieve a self-balancing attitude adjustment, so as to keep the sampling position stable. With the rotation of the suspended carrying platform 2 relative to the suspension cabin main body 1 as well the use of the balancing device, a self-balancing attitude adjustment can be quickly achieved to maintain a horizontally stable state, so as to ensure the reliability of testing by the water sample testing device, realize on-water attitude adjustment and stable position control, and prevent the disturbance on the water surface from affecting the monitoring and assessment result.

A third feature of the device lies in that when the water quality assessment model generates and outputs the monitoring and assessment result, the suspension cabin main body 1 can give a visual warning according to the status of the water quality of the water region. The visual warning may include but is not limited to three warning fluorescent signals corresponding to different states from the outer cover, which respectively indicate that the water quality does not reach the discharging standard, the water quality index is about to reach the discharging standard, and the water can be discharged. This can visually reflect the results of water quality monitoring and assessment in the water region, give a warning when the water quality does not meet the quality standard, and quickly send warning information to the outside, thereby improving the early warning monitoring and management efficiency.

A fourth feature of the device lies in that with the arrangement of the intelligent gateway of the communication and control system and the communication antenna 33, communication between a satellite and the Internet of Things device including the PC-side host computer can be realized to obtain location information and transmit data in real time, thereby achieving planning of the sampling and cruising path, self-cruising and fixed-point suspension, as well as remote monitoring and management of the monitoring and assessment result of the water region.

(2) The monitoring method based on the suspended self-balancing self-cruising online water quality monitoring device automatically plans a W-shaped water sampling and cruising path, thereby solving the problem of continuously optimizing the monitoring of different waters. According to the satellite topography data of the water region, the positions and number of the shore points are optimized based on whether the area of the region defined by lines connecting every three adjacent shore points is less than or equal to the characteristic region threshold, to optimize the W-shaped water-region cruising path to be a shortest path under a condition that the sampling points fully represent an overall status of the water region, and facilitate the continuous mobile monitoring of different water regions. The monitoring is implemented automatically, which improves the precision of water quality monitoring to a certain extent, and greatly reduces the costs of monitoring the water region.

(3) The assessment method based on the monitoring method overcomes the incomprehensiveness of water body assessment. The multi-parameter online monitoring results of all the sampling points in the water region and the assessment factor are fitted to obtain comprehensive assessment vector data of the water region, a water quality assessment result of the water region is generated and outputted according to a result of comparison between the vector and the water quality assessment criterion, and the overall status of the water region is comprehensively assessed and analyzed. Whereby, the assessment of water bodies is implemented automatically and more comprehensively and accurately.

To sum up, according to the technical solutions of the present invention, with the suspended self-balancing self-cruising online water quality monitoring device, the online water quality monitoring method, and the online water quality assessment method, a path and sampling points are automatically planned, precise positioning and self-cruise monitoring are achieved, the device remains stable throughout the monitoring process, and self-balancing of the device is not affected by the disturbance on the water surface, thereby improving the monitoring and assessment precision, and greatly reducing the cost of water-region monitoring and assessment, making the device especially suitable for the monitoring and assessment of water quality in different water regions that have flowing water and require continuous monitoring.

It should be understood that although this specification is described in accordance with various embodiments, it does not mean that each embodiment only contains an independent technical solution. The description in the specification is only for clarity, and those skilled in the art should regard the specification as a whole, and the technical solutions in the embodiments can also be appropriately combined to form other implementations that can be understood by those skilled in the art.

The detailed descriptions listed above are merely specific illustrations of feasible embodiments of the present invention, and the protection scope of the present invention is not limited thereto. Equivalent embodiments or changes can be made without departing from the technical spirit of the present invention, which are all embraced in the protection scope of the present invention.

What is claimed is:

1. A suspended self-balancing self-cruising online water quality monitoring device, comprising a suspension cabin main body, a communication and control system, and a suspended carrying platform located inside the suspension cabin main body and configured for self-balancing attitude adjustment, wherein a plurality of suspension feet are arranged outside the suspension cabin main body; each of the suspension feet is configured to drive the suspension cabin main body to perform self-balancing attitude adjustment, cruising, or fixed-point suspension; a water sample testing device is arranged on the suspended carrying platform; and the communication and control system is configured to receive a satellite signal, plan a W-shaped water-region cruising path and sampling points on the W-shaped water-region cruising path according to a topography of a water region, control an operation of the suspension feet, control an attitude adjustment of the suspended carrying platform, control the water sample testing device to test a quality of water outside the suspension cabin main body along the sampling points online, assess a water quality of the water region according to an online testing result of the water sample testing device, and output a monitoring and assessment result.

2. The suspended self-balancing self-cruising online water quality monitoring device according to claim 1, wherein the suspension cabin main body comprises a suspended bottom compartment having a changeable center of buoyancy and located at a bottom of the suspended carrying platform, and a cover connected to the suspended bottom compartment and located outside the suspended carrying platform; and the cover is configured to display a corresponding visual warning signal according to the monitoring and assessment result from the communication and control system.

3. The suspended self-balancing self-cruising online water quality monitoring device according to claim 1, wherein the communication and control system comprises an external controller; the external controller comprises a Global Positioning System (GPS) module, a communication module, and an external core controller configured to coordinate an operation of the external controller; the GPS module is configured to communicate with a satellite through the communication module to feed back a location information; the external core controller integrates an automatic W-shaped water-region cruising path planning algorithm; and the automatic W-shaped water-region cruising path planning algorithm is configured to solve and optimize the W-shaped water-region cruising path and the sampling points for the water sample testing device according to the location information from the GPS module, and optimize the W-shaped water-region cruising path to be a shortest path under a condition that the sampling points represent an overall status of the water region.

4. The suspended self-balancing self-cruising online water quality monitoring device according to claim 3, wherein the external controller comprises a first gyroscope and a first driving module; the first gyroscope is configured to analyze an attitude of the suspension cabin main body; the first driving module is configured to drive the suspension feet to perform attitude adjustment and operate; and the external core controller is configured to control an operation of the first driving module according to a result of the automatic W-shaped water-region cruising path planning algorithm and/or an attitude feedback from the first gyroscope.

5. The suspended self-balancing self-cruising online water quality monitoring device according to claim 4, wherein each of the suspension feet comprises a suspension leg and an ellipsoid suspension body; one end of the suspension leg is connected to the suspension cabin main body, and the other end of the suspension leg is connected to the ellipsoid suspension body; the suspension leg comprises at least one joint; the joint is provided with a first driving mechanism connected to the first driving module; the first driving mechanism is configured to drive the joint to move relatively; a second driving mechanism is arranged between the ellipsoid suspension body and the suspension leg; and the second driving mechanism is connected to the first driving module and is configured to drive the ellipsoid suspension body to rotate relative to the suspension leg.

6. The suspended self-balancing self-cruising online water quality monitoring device according to claim 1, wherein the suspended carrying platform is rotatable relative to the suspension cabin main body; a balancing device configured to drive the suspended carrying platform to self-balance is arranged on the suspended carrying platform; the communication and control system comprises an internal controller; the internal controller comprises a second gyroscope, a second driving module, and an internal core controller configured to coordinate an operation of the internal controller; the second gyroscope is configured to analyze an attitude of the suspended carrying platform; the second driving module is configured to drive the balancing device and the water sample testing device to operate; the internal core controller is configured to control the second driving module to drive the balancing device according to an attitude feedback from the second gyroscope; a water-region water quality assessment model is integrated on the internal core controller; and the water-region water quality assessment model is configured to assess the water quality of the water region according to the online testing result of the water sample testing device and generate the monitoring and assessment result.

7. The suspended self-balancing self-cruising online water quality monitoring device according to claim 6, wherein the suspended carrying platform is rotatably connected to the suspension cabin main body through a spherical hinge connecting rod; the spherical hinge connecting rod comprises a load-bearing rod, a spherical hinge ball shell, a spherical hinge ball, and a carrying-platform connecting rod; one end of the load-bearing rod is connected to a top of the suspension cabin main body, and the other end of the load-bearing rod is connected to the spherical hinge ball shell; the spherical hinge ball is arranged inside the spherical hinge ball shell and is rotatably fitted to an interior of the spherical hinge ball shell; one end of the carrying-platform connecting rod is connected to the spherical hinge ball, and the other end of the carrying-platform connecting rod is connected to the suspended carrying platform or the internal controller; the balancing device comprises two balancing motors oppositely arranged on the suspended carrying platform; a motor shaft of each of the balancing motors is connected to a balancing flywheel;
the suspension cabin main body comprises a fluorescent cover; a light-emitting diode (LED) warning light strip configured to output a corresponding light source according to the monitoring and assessment result from the communication and control system is arranged on the internal controller; the fluorescent cover is configured to emit a corresponding visual warning fluorescent signal under an excitation of the LED warning light strip; and the internal controller communicates with an Internet of Things device and outputs the monitoring and assessment result.

8. The suspended self-balancing self-cruising online water quality monitoring device according to claim 1, wherein the water sample testing device comprises a water inlet pipeline, a testing chamber, a water quality sensor array, a water level sensor group, and a water outlet pipeline; the water inlet pipeline is communicated with an outside of the suspension cabin main body and the testing chamber; the testing chamber is configured to store a filtered water sample inputted through the water inlet pipeline; the water quality sensor array is configured to test the filtered water sample in the testing chamber and transmit the online testing result comprising at least one type of parameters or at least one parameter; the water level sensor group is configured to feed back a water level of the filtered water sample in the testing chamber to the communication and control system; the water outlet pipeline is communicated with the outside of the suspension cabin main body and the testing chamber; and the communication and control system is configured to control on/off of a water intake through the water inlet pipeline and on/off of a water output through the water outlet pipeline, and control an operation of the water quality sensor array.

9. The suspended self-balancing self-cruising online water quality monitoring device according to claim 1, wherein a power supply system is arranged on the suspension cabin main body; the power supply system comprises a solar panel arranged on the suspension cabin main body; and the communication and control system is configured to control the power supply system to supply power to the communication and control system, the suspension feet, and the water sample testing device.

10. An online water quality monitoring method based on the suspended self-balancing self-cruising online water quality monitoring device according to claim 1, comprising:
S1: receiving, by the communication and control system, the satellite signal and obtaining a satellite topography data of the water region;
S2: calculating a longest diameter of the topography of the water region according to the satellite topography data, and setting two ends of the longest diameter as a starting shore point and a destination shore point, respectively;
S3: selecting several shore points on two sides of the longest diameter, and numbering the shore points alternately on the two sides starting from the starting shore point to the destination shore point, so that the shore points with adjacent numbers are connected to form an integer number of W-shaped patterns;
S4: calculating a characteristic region threshold according to the satellite topography data, wherein the characteristic region threshold is an upper limit of an area characterizing a property of the water region; optimizing positions and a number of the shore points based on whether an area of a region defined by lines connecting every three adjacent shore points of the shore points is less than or equal to the characteristic region threshold, and generating the W-shaped water-region cruising path planned according to the topography of the water region;
S5: selecting the sampling points on the W-shaped water-region cruising path;
S6: controlling, by the communication and control system, the suspension cabin main body to cruise along the sampling points;
S7: at each of the sampling points, controlling the suspension cabin main body to be suspended fixedly, and implementing a self-balancing attitude adjustment of the suspension cabin main body and the suspended carrying platform; and
S8: controlling the water sample testing device to test the quality of the water outside the suspension cabin main body online, generating and outputting an online monitoring result of the water quality of the water region.

11. The online water quality monitoring method according to claim 10, wherein the step S1-step S6 are based on an automatic W-shaped water-region cruising path planning algorithm; in the step S4, an area of a triangle defined by lines connecting every three adjacent shore points of the shore points is numbered sequentially based on the automatic W-shaped water-region cruising path planning algorithm, wherein the area of the triangle is Sⱼ, j is an integer from 1 to 4n−1, n is an integer equal to or greater than 1; a calculation formula of the characteristic region threshold M is M=3S_{water-region}/H*(4n−1), H=hₘ/hₗ, wherein S_{water-region} represents a total area of the water region in the satellite topography data in the step S1, hₘ represents a water depth at a deepest position of the water region in the satellite topography data in the step S1, and hₗ is a water depth at a shallowest position of the water region in the satellite topography data in the step S1; and the positions and the number of the shore points are optimized with all Sⱼ≤M to generate the W-shaped water-region cruising path planned according to the topography of the water region.

12. An online water quality assessment method based on the online water quality monitoring method according to claim 10, comprising:
S9: setting, by the communication and control system, a water quality assessment criterion and an assessment factor;
S10: constructing a matrix comprising testing data of each of the sampling points according to the online monitoring result of the online water quality monitoring method;
S11: calculating a vector according to the matrix and the assessment factor; and
S12: generating and outputting a water quality assessment result of the water region according to a result of comparison between a norm of the vector and the water quality assessment criterion.

13. The online water quality assessment method according to claim 12, wherein the step S9-step S12 are based on a water-region water quality assessment model; a calculation formula of the assessment factor N of the water-region water quality assessment model in the step S9 is N=e*(1/k), wherein e represents a unit vector, and k represents a vector dimension of the matrix in the step S10; in the step S10, testing data obtained by the water sample testing device at an i^{th} sampling point is used as an m-dimensional vector Xᵢ, and a matrix M is constructed using k-dimensional Xᵢ to calculate a vector Xₒ, wherein a calculation formula of Xₒ is Xₒ=M*N; and in the step S12, a calculation formula of the norm is ∥Xₒ∥₂=λ, and the water quality assessment result of the water region is generated by comparing k with the water quality assessment criterion.

14. The online water quality monitoring method according to claim 10, wherein the suspension cabin main body comprises a suspended bottom compartment having a changeable center of buoyancy and located at a bottom of the suspended carrying platform, and a cover connected to the suspended bottom compartment and located outside the suspended carrying platform; and the cover is configured to display a corresponding visual warning signal according to the monitoring and assessment result from the communication and control system.

15. The online water quality monitoring method according to claim 10, wherein the communication and control system comprises an external controller; the external controller comprises a Global Positioning System (GPS) module, a communication module, and an external core controller configured to coordinate an operation of the external controller; the GPS module is configured to communicate with a satellite through the communication module to feed back a location information; the external core controller integrates an automatic W-shaped water-region cruising path planning algorithm; and the automatic W-shaped water-region cruising path planning algorithm is configured to solve and optimize the W-shaped water-region cruising path and the sampling points for the water sample testing device according to the location information from the GPS module, and optimize the W-shaped water-region cruising path to be a shortest path under a condition that the sampling points represent an overall status of the water region.

16. The online water quality monitoring method according to claim 15, wherein the external controller comprises a first gyroscope and a first driving module; the first gyroscope is configured to analyze an attitude of the suspension cabin main body; the first driving module is configured to drive the suspension feet to perform attitude adjustment and operate; and the external core controller is configured to control an operation of the first driving module according to a result of the automatic W-shaped water-region cruising path planning algorithm and/or an attitude feedback from the first gyroscope.

17. The online water quality monitoring method according to claim 16, wherein each of the suspension feet comprises a suspension leg and an ellipsoid suspension body; one end of the suspension leg is connected to the suspension cabin main body, and the other end of the suspension leg is connected to the ellipsoid suspension body; the suspension leg comprises at least one joint; the joint is provided with a first driving mechanism connected to the first driving module; the first driving mechanism is configured to drive the joint to move relatively; a second driving mechanism is arranged between the ellipsoid suspension body and the suspension leg; and the second driving mechanism is connected to the first driving module and is configured to drive the ellipsoid suspension body to rotate relative to the suspension leg.

18. The online water quality monitoring method according to claim 10, wherein the suspended carrying platform is rotatable relative to the suspension cabin main body; a balancing device configured to drive the suspended carrying platform to self-balance is arranged on the suspended carrying platform; the communication and control system comprises an internal controller; the internal controller comprises a second gyroscope, a second driving module, and an internal core controller configured to coordinate an operation of the internal controller; the second gyroscope is configured to analyze an attitude of the suspended carrying platform; the second driving module is configured to drive the balancing device and the water sample testing device to operate; the internal core controller is configured to control the second driving module to drive the balancing device according to an attitude feedback from the second gyroscope; a water-region water quality assessment model is integrated on the internal core controller; and the water-region water quality assessment model is configured to assess the water quality of the water region according to the online testing result of the water sample testing device and generate the monitoring and assessment result.

19. The online water quality monitoring method according to claim 18, wherein the suspended carrying platform is rotatably connected to the suspension cabin main body through a spherical hinge connecting rod; the spherical hinge connecting rod comprises a load-bearing rod, a spherical hinge ball shell, a spherical hinge ball, and a carrying-platform connecting rod; one end of the load-bearing rod is connected to a top of the suspension cabin main body, and the other end of the load-bearing rod is connected to the spherical hinge ball shell; the spherical hinge ball is arranged inside the spherical hinge ball shell and is rotatably fitted to an interior of the spherical hinge ball shell; one end of the carrying-platform connecting rod is connected to the spherical hinge ball, and the other end of the carrying-platform connecting rod is connected to the suspended carrying platform or the internal controller; the balancing device comprises two balancing motors oppositely arranged on the suspended carrying platform; a motor shaft of each of the balancing motors is connected to a balancing flywheel;
the suspension cabin main body comprises a fluorescent cover; a light-emitting diode warning light strip configured to output a corresponding light source according to the monitoring and assessment result from the communication and control system is arranged on the internal controller; the fluorescent cover is configured to emit a corresponding visual warning fluorescent signal under an excitation of the LED warning light strip; and the internal controller communicates with an Internet of Things device and outputs the monitoring and assessment result.

20. The online water quality monitoring method according to claim 10, wherein the water sample testing device comprises a water inlet pipeline, a testing chamber, a water quality sensor array, a water level sensor group, and a water outlet pipeline; the water inlet pipeline is communicated with an outside of the suspension cabin main body and the testing chamber; the testing chamber is configured to store a filtered water sample inputted through the water inlet pipeline; the water quality sensor array is configured to test the filtered water sample in the testing chamber and transmit the online testing result comprising at least one type of parameters or at least one parameter; the water level sensor group is configured to feed back a water level of the filtered water sample in the testing chamber to the communication and control system; the water outlet pipeline is communicated with the outside of the suspension cabin main body and the testing chamber; and the communication and control system is configured to control on/off of a water intake through the water inlet pipeline and on/off of a water output through the water outlet pipeline, and control an operation of the water quality sensor array.
